(19) United States
(12) Patent Application Publication
(10) Pub. No.: US 2003/0022206 A1
(43) Pub. Date: Jan. 30, 2003

(54) Method of characterizing a homeopathic preparation

(76) Inventors: Robert A. Kay, Brea, CA (US); Danute M. Bankaitis-Davis, Longmont, CO (US)

Correspondence Address: Ronald J. Baron, Esq., HOFFMANN & BARON, LLP, 6900 Jericho Turnpike, Syosset, NY, 11791, US

(21) Appl. No.: 10/146,661

(22) Filed: May 15, 2002

Related U.S. Application Data
(60) Provisional application No. 60/291,592, filed on May 16, 2001.

(51) Int. Cl. C12Q 1/68; A61K 35/78; A61K 33/00

(52) U.S. Cl. 435/6; 424/600; 424/725

(57) ABSTRACT

A method is provided for obtaining a measure characteristic of a homeopathic preparation that affects the expression of at least one gene, the method comprising contacting the preparation with a gene in a gene expression system; and measuring the level of expression of the gene; wherein the level of expression constitutes the measure characteristic of the contents of the preparation.

### BACKGROUND OF THE INVENTION

This invention relates to obtaining a measure characteristic of a homeopathic preparation by measuring the bioactivity. More specifically, this invention provides novel methods of a reproducible, qualitative, biologically relevant characterization of homeopathic preparations useful in all aspects of development, manufacture and use, requiring characterization. The primary and immediate uses will be for research and improving the quality control of homeopathic preparations by contacting the drug with a gene expression system and measuring the amount of gene expression.

Although some of the basic ideas of homeopathy can be dated back to ancient Greek and Roman times, homeopathy as it is presently known is considered to have been originated by Samuel Christian Hahnemann (1755-1843).

Hahnemann, a chemistry and medical student at the Universities of Leipzig, Berlangen, and Vienna (ultimately qualifying as a doctor in 1779), became increasingly disillusioned with convienential medical practice and eventually gave up being a doctor to work as a translator. During his work as a translator, Hahnemann came across a passage about the ability of quinine purified from the bark of the cinchona tree to treat malaria. Upon further investigation, Hahnemann decided to dose himself with quinine and record the results. Surprisingly, Hahnemann discovered that he began to develop the symptoms of malaria.

This experience led Hahnemann to the principle of “like can cure like,” now sometimes referred to as the “law of similars.” Hahnemann began to test or “prove” other materials on healthy individuals. The substances were then administered to the sick to determine whether or not they benefited. In 1810, Hahnemann set out his principles in the Organon of Rationale Medicine, and two years later began teaching homeopathy at the University of Leipzig.

Because some of the substances used to derive the homeopathic preparations by Hahnemann were poisonous, he gave them to patients in very small, dilute doses. However, side effects were still observed. Hahnemann therefore derived a two-step process whereby he greatly diluted each remedy and vigorously shook the remedy at each stage of the dilution. Hahnemann observed that the diluted and shaken medicines avoided unwanted side effects and seemed to act faster and more effectively than the more concentrated solutions.

According to the Homeopathic Pharmacopoeia of the United States, “Homeopathy is the art and science of healing the sick by using substances capable of causing the same symptoms, syndromes and conditions when administered to healthy people.” Any substance may be considered a homeopathic preparation if it is known to mimic the symptoms, syndromes or conditions in healthy people to whom it is administered.

A complication in the field of homeopathy is the lack of reproducible quality control for in-process or finished homeopathic products. In fact, the Homeopathic Pharmacopoeia of the United States in its Official Compendium, pg. 63 (July 1992), states that due to the “infinitesimal doses of active ingredients . . . it is impossible to carry out analytic controls of the finished products.”

Homeopathic industries are coming under increasing pressure to upgrade their quality control practices. However, this has proven difficult in light of the nature of homeopathic preparations, and inability of standard chemical based test method to characterize in-process or finished products.

Various types of tests have been instituted in an attempt to assure the consistent quality of homeopathic preparations; including a variety of chemical analyses. Recently, high performance liquid chromatography (HPLC) has been used to profile marker molecules in homeopathic preparations. However, there are problems with this approach, in that the amount of a chemical is not necessarily proportional to its biological potency.

DE 4214405 (1993) discloses a measurement and display system for analysis of the effects of homeopathic substances by measuring electric voltage and current variations with time by mean of electrodes immersed in the solution.

Russian Patent No. RU 2112976 C1 (1997) describes a method for determining the quality of homeopathic preparations and a device for its implementation. The method includes illuminating the compositions with coherent, linearly polarized, optical radiation. The scattered radiation is then recorded, analyzed and compared with the corresponding optical characteristics of a reference sample. A multiple dilution of the homeopathic preparation is used as an investigated medium and is placed in a constant magnetic field. The scattered radiation is recorded by temporary storage of the intensity of its polarization component under conditions of optical mixing from different points of the investigated medium. An analysis is accomplished with a calculation of the frequency spectrum of super slow fluctuations in intensity of the scattered radiation and its comparison with reference samples.

Irish Patent Application No. 940085 (1995) describes a method for testing the quality of a homeopathic preparation by subjecting a test sample of the homeopathic preparation to an alternating current electrical signal, varying the frequency of the electrical signal, and monitoring the conductance, inductive characteristic, and/or the capacitive characteristic, and recording the frequency of the electrical signal at which a change in the monitored characteristic occurs.

Irish Patent Application No. 940084 describes another method for testing a homeopathic preparation. This method includes placing a test sample of the homeopathic preparation in an electro-plasma field so that the field in the near vicinity of the test homeopathic preparation fluoresces. A photograph is then taken of the fluorescing electro-plasma field and the film is developed to provide a negative of the image. Laser light is then passed through the negative and the image formed by the laser light is then analyzed.

German Patent No. DE4231385 (1994) discloses a method for testing a substance in high dilution. The substance is placed as a liquid in a dish and a reference substance with the same solvent and the same dilution is placed in an identical dish. Each dish is at a branch of a bridge and both sides are energized simultaneously electrically, electromagnetically or magnetically at different frequencies. The difference of the energizing for each dish is established and evaluated for each frequency value.

These known optical and electrical methods described above to ensure the quality of homeopathic preparations have also proven ineffective. Therefore, there remains a need for a reliable method for obtaining a measure characteristic of the contents of a homeopathic preparation.

### SUMMARY OF THE INVENTION

The present invention is a method for obtaining a measure characteristic of a homeopathic preparation or solution by contacting the preparation with at least one gene in a gene expression system and measuring the level of expression of the at least one gene. Preferably this method is used for determining the characteristics, e.g., qualitative and quantitative indicator of potency and purity, of a homeopathic preparation. By obtaining a measure characteristic, a method of ensuring quality control of a homeopathic preparation is also provided. Thus, in assessing the quality of such preparations, more than one determination of the average level of the gene expression can be made in a system and then contacting subsequent preparations and comparing same to a standard developed by the previous assessments.

This invention, which measures effect on a biological system, substantially differs from the above mentioned methods that rely on the reaction of a chemical(s) or an outside force and measures the affect.

As a result of the present invention, predictability and quality control can be exercised in the manufacture and sale of homeopathic preparations. Gene expression can detect and quantify biological effect of homeopathic products without the need to suppose a method of action or know active composition (as in nuclear magnetic resonance, chemical analysis, etc.). Alone this test can be uses as a measure of consistency without considering issued of efficacy. As measure characteristics are developed and associated with clinical studies, they can be applied to yield a quantitative pharmacological probability.

### BRIEF DESCRIPTION OF THE DRAWINGS

FIG. 1 depicts a gene expression profile for diluent response in unstimulated whole blood.

FIG. 2 depicts a gene expression profile for arnica response in unstimulated whole blood: Boericke & Tafel Homeopathic Arnica@D3, D6, D12, D30, D200.

FIG. 3 depicts a gene expression profile for arnica response in unstimulated whole blood: Boericke & Tafel Homeopathic Arnica@D3, D6, D12, d30 and D200 compared to LPS.

FIG. 4 depicts a gene expression profile for controls in unstimulated whole blood: USP water and lactose tablet.

FIG. 5 depicts a gene expression profile for arnica tablet lot comparison@200C in unstimulated whole blood: Hahnemann lots Q.01.02.23, 2CM23Q and 1972.

FIG. 6 depicts a gene expression profile for arnica tablet manufacturer comparison@200C in unstimulated whole blood: Boiron, Hahnemann and Standard.

FIG. 7 depicts a gene expression profile for Boericke & Tafel Arnica solution comparison in unstimulated whole blood: D30) succussed versus D30 unsuccussed.

FIG. 8 depicts a gene expression profile for Boericke & Tafel Arnica solution comparison in unstimulated whole blood: D200 succussed versus D200 unsuccussed.

### DETAILED DESCRIPTION OF THE INVENTION

The inventors have discovered a novel method for obtaining a measure characteristic of a homeopathic preparation which affects the expression of at least one gene.

Homeopathic preparations are defined herein by the way they are prepared. Homeopathic preparations include homeopathic “Mother Tinctures”. Homeopathic preparations also include attenuated preparations which are prepared by diluting the mother tincture to a certain potency, and succussing (vigorously shaking) the solution during each iteration of the dilution (attenuation) process.

Homeopathic preparations thus prepared by definition can not be confused with herbal compositions. Herbal compositions are not prepared using the specific homeopathic preparation methods including dilution and succussing. Further, homeopathic preparations operate under the principle of “like can cure like,” i.e. the “law of similars”. The activity of homeopathic preparations on an individual is based on an active ingredient known to mimic the symptoms the solution is designed to treat. Herbal compositions on the other hand operate as do conventional drugs, where the active ingredient works in a specific biochemical manner

Recognizing the difference between homeopathic preparations and herbal compositions, the government has promulgated different regulations for the two substances. Homeopathic preparations, which are listed in the Homeopathic Pharmacopeia of the United States (HPUS) fall within the jurisdiction of the FDA pursuant to the Federal Food, Drug and Cosmetic Act (FDCA). See 21 U.S.C. §321(g).

In the United States, homeopathic preparations are recognized as drugs by the Food Drug and Cosmetic Act—SEC. 210.[321](g)(1)(A) hence are regulated primarily by the Food and Drug Administration and the Federal Trade Commission under the guidelines of CFR 21 Homeopathic preparations are also widely recognized internationally as drugs by the specific national drug regulations. In the US Drugs are specifically differentiated from foods and dietary supplements 210.[321](g)(C). Homeopathic products are required to comply with US drug Good Manufacturing Practices and production and labeling standards are set forth in CFR 21 as well as by reference to the Official Compendia, the HPUS. Although some of the drug manufacturing requirements are exempted for homeopathic drugs in consideration of the inability of final product testing (Sec. 211.137 Expiration Dating) it does require a modified stability testing program and general compliance to Good Manufacturing Practices (CFR 21 Part 211) both of which would be greatly enhanced by the ability to do final product testing rather than relying solely on process control as is current practice.

Herbal preparations differ significantly from homeopathic preparations from a regulatory perspective as well as medically. The vast majority of herbal preparations are classified as Dietary Supplements placing them under the regulatory authority of the Dietary Supplement Health and Education Act (DSHEA) of 1994. Essentially this regulates supplements as foods with additional allowances for a type of limited claim called a “structure function claim”.

Homeopathic preparations are made from botanical, zoological or chemical substances, (“chemical substances”—defined as mineral or organic chemical products, complex substances of natural origin, or products defined only by their manufacturing process) known as crude drug. These crude drugs are prepared into an initial extraction called the mother tincture, also know as a 1X preparation, though process of maceration, solvent extraction, incubation, fermentation or any other method set forth in an appropriate compendium such as the HPUS. For soluble substances the crude drug is put into solution, by a specific process set forth in the Compendium, generally consisting of 1 part theoretical dry crude drug (weight of the dry drug with no allowance for naturally occulting water) to 9 parts solvent without succussion (although it is agitated to facilitate extraction).

If the substance for deriving the remedy, such as a mineral or metal, is not soluble in water or alcohol, a specific method of trituration is used. In this method the substance is continually ground until it becomes soluble.

The mother tincture (i.e. 1X) can then be diluted to prepare different potencies. One part of the mother tincture is diluted in a specific amount of solvent, usually 9, 99, or 49,999 parts to one of the mother tincture. The resulting solution is then shaken forcefully along all spatial axes and primarily along two axes, e.g., x and y axes. This forceful shaking is called succussing. The number of succussions is definite depending on the remedy being prepared. There are usually between approximately between 40 and 100 succussions at each potency level. One part of this solution is then diluted and succussed similarly, and the process is continued until a specified number of dilution/succussions have been attained.

A mother tincture that has undergone the process of succession is said to be attenuated. Attenuation is the process where by the mother tincture is chemically diluted to 1/10, 1/100 or 1/50,000 of the original chemical composition and succussed. According to the theory of homeopathy, although the chemical concentration of the mother tincture is decreased the pharmacological potency increases. Any dilution beyond 1⁻²⁴ (or in homeopathic terminology 24X or 12C) theoretically will have no molecules of the original substance left (Avogadro's number). Since 30X and 200X and greater attenuations are common in homeopathic practice, standard tests for chemical compositions are not appropriate.

An alternative method for preparing the dilution (attenuation) is the Korsakoff method. When using the Korsakoff method, the solution from the previous potency is poured-out, leaving a film on the walls of the vial. New solvent is then added to the same vial, succussed, and diluted as set forth above.

The dilution (attenuation) of the mother tincture is performed according to one of two different scales, the decimal (X) scale and centesimal (C) scale. The decimal scale is based on a dilution of 1/10. The first 1/10 potency is a 1/10 dilution (mother tincture) or a ratio of 1 part theoretical dry crude drug to 9 parts total solvent. The second dilution (Always a dilution with no crude drug) (1/10×1/10=1/100) is called a 2× potency.

The centesimal scale is the most commonly used. The centesimal scale is based on a dilution of 1/100. For example, to produce a 1C potency of a remedy, one part of the mother tincture is added to 99 parts of vehicle (e.g. alcohol/water solution) and succussed. To produce a 2C potency, one part of the 1C mixture is added to 99 parts of an alcohol/water solution and succussed.

For example, studies have been conducted where doses of histamine were administered that were diluted 1:100 15 to 19 times. The administration of this homeopathic preparation containing histamine as its active ingredient were reported to demonstrate significant effects on inhibiting degranulation of basophilis (p<0.0001). See, Ullman, Dana, “The Thermodynamics of Extremely Diluted Solutions, New Evidence for Homeopathic Microdoses,” Homeopathic Educational Services (1999).

Homeopathic preparations pose unique problems with regard to quality control and ensuring consistency between the same homeopathic preparations. Applicants have unexpectedly found that gene expression can be used to obtain a measure characteristic for homeopathic preparations.

The gene can be any gene whose expression is affected by the homeopathic preparation. The effect can be any qualitative or quantitative changes of expression levels of the gene. Examples of genes that be used include but are not limited to: IL-1alpha; IL-1beta; IL-2; IL-4; IL-6; IL-7; IL-8; IL-10; IL-12p40; IL-15; IL-18; GM-CSF; IFN-gamma; TGF-beta; TNF-alpha; ICE; c-juh; MMP-9; u-pa; HSP-70; cre; ICAM; Cox-2; Cox-1, or a combination thereof.

The method includes contacting the homeopathic preparation with a gene in a gene expression system. In one embodiment, the homeopathic preparation has not been diluted to a level beyond 12c or 24x. The gene expression system refers to any system in which genes may be expressed. The gene expression system may be in vitro, ex vivo or in vivo. In vitro systems typically include cultured samples. Ex vivo systems typically include cells or organs removed from the living animal. In vivo systems include living animals. Thus, the gene expression system includes, but is not limited to, any cell, tissue, organ, whole organism or in vitro system that expressed the gene when contacted with the composition.

After contacting the gene expression system with the composition, the level of gene expression is measured. Methods for measuring the level of gene expression are well known in the art. Examples of particularly useful technologies for gene expression include, microarray technology, reverse transcriptase polymerase chain reactions (PCR), differential display, large-scale sequencing of expressed sequence tags (ESTs), serial analysis of gene expression (SAGE), and Western immunoblot or 2D, 3D study of proteins. One skilled in the art of molecular marker technology is familiar with the methods and uses of such technology (see, e.g.,) Bernard R. Glick and Jack J. Pasternak, Molecular Biotechnology. Principles and Applications of Recombinant DNA, Second Edition, ASM Press (1998); Mathew R. Walker and Ralph Rapley, Route Maps in Gene Technology, Blackwell Science (1997); Roe et al., DNA Isolation and Sequencing, John Wiley & Sons (1996) James D. Watson et al., Recombinant DNA, Second Edition, Scientific American Books (1992)).

Source Precision Medicine (Boulder, Colo.) offers a particularly useful method for measuring gene expression initiated by contact with a particular composition. This method is set forth in WO 01/25473 and is incorporated herein by reference as if set forth in full. Source Precision Profiles™ are gene expression profiles developed for compositions through differential display, semi-quantitative screening techniques (microarray), and real time PCR analysis. The profiles typically consist of from 6 to 48 selected gene loci that are measured by quantitative PCR to give a standardized, calibrated output of differential gene expression for selected markers. The profiles generated can then be compared.

Source Precision Medicine has developed a number of techniques called “Precision Profiles™.” These profiles are genetic expression fingerprints obtained from a specific cell type or from whole blood, representing in essence a dynamic measure of how an individual cell or a group of cells working together express their genetic responses. A profile can demonstrate how a selected group of genes respond to a particular agent.

Since the minute genetic responses of the cells are very hard to detect, Source Precision has developed an amplification method that measures the abundance of the messenger RNA (mRNA) expressed by 6 to 48 gene loci in the DNA. These loci have been pre-selected (by reviewing existing medical and research literature on gene expression) t reflect a pattern that has been linked to a therapeutic area of interest. Thus a precision profile consists of a true differential gene expression of a particular cell type or in blood.

For example, in Source Precision's inflammatory gene panel, they have selected 24 gene loci in THP-1 cells (a human cultured cell line) that regulate the expression of cytokines, proteases and cell cycle regulatory components. A group of profiles can be generated by testing a known concentration of control (vehicle) and various concentrations of the sample added to the culture. The cells are harvested and the messenger RNA extracted and amplified by using competitive polymerase chain reaction methods. The profiles are generated by looking at these pieces of mRNA. Once profiles are graphed, they can be contrasted. The profiles provide identity of the component (specific cytokine or cell factor coded in the mRNA) and the amount it has been generated at a certain period of time.

The approach by Source Precision identifies reproducible patterns of variation of gene expression that are informative by virtue of the degree of variation between a sample and a baseline. The amount of gene expression product (for example RNA transcript) produced by a gene locus in a cell under certain circumstances is measured and then stored as a value in a first profile data set. This value is calibrated with respect to a second value (a baseline profile data set) to provide a member of a calibrated profile data set. The values recorded for the profile data set, relying on a particular baseline data set to produce a calibrated data set become part of the descriptive record any or all of which can be stored in a database which may be accessed through a global network such that any new data in the form of a profile data set or a calibrated profile data set measured at any global location can be directly compared to an archive of descriptive records including calibrated profile data sets and baseline data sets so as to extend the stored library of profiles and provide predictive or diagnostic data about a particular biological condition or agent.

Steps in selecting constituents in the Source Precision gene panel include searching publicly available medical literature for RNA or proteins or sets of RNAs or proteins that directly or indirectly vary with a particular biological condition. For the method of the invention, a gene panel would preferably be chosen such that the expression of the genes vary with a biological condition that is targeted therapeutically by the homeopathic preparation. For example, if the homeopathic preparation is an anti-inflammatory, a preferred gene expression panel would include genes that vary expression in response to the biological condition of inflammation.

For purposes of illustrating a signature panel, constituents of a panel for measuring inflammation have been provided by Source Precision that are informative with respect to a particular biological condition. For example, they have used a panel for inflammation that has 6 constituents—Il-1a, Il-6, Il-8, Il-18, GMCSF and IFN-g in FIG. 18(a)-(e) to determine the response of 5 subjects to varying concentrations of drugs. This group of constituents is a subset of a larger panel of inflammation related gene loci where the Inflammatory Panel includes Il-a, Il-b, Il-2, Il-3, Il-4, Il-6, Il-7, Il-8, Il-10, Il-12p40, Il-15, Il-15, Il-18, GM-CSF, Ifn-gamma, TGF-b, cox-2, ICE, MMP-9, ICAM, TNF-a and TNF-b.

A panel containing up to 100 constituents may be selected. According to the condition being examined, just a small subset of the panel constituents may be informative. In determining membership of the panel of genes, it is not necessary for the panel to be an exhaustive selection. Rather, it is desired to obtain from the panel an expression profile that discriminates consistently with respect to the targeted sample. Moreover, a panel is not necessarily selected according to an expected profile of gene expression in cells that directly respond to a biological effect.

The number of constituents in a panel can vary. Although a panel may be as large as 100 constituents, it is desirable for a particular panel to have no more than 24 constituents, more particularly, less than 12 constituents. For example, subsets of no more than 8 genes have been used that may be derived from a larger panel but which are sufficiently informative to effectuate discrimination. The number of constituents in a panel for which expression is monitored may vary widely depending on the context.

Panels of varying sizes may be utilized as necessary and subsequent refinements in methodology may lead to selection of subsets having panels as large as 15 genes or 12 genes or as small as 6, 5, 4, 3 or 2 genes.

Source Precision has developed least 4 different panels which may be used separately or together. These panels are an inflammatory panel (TNF-a, Il-1b, ICAM, Il-8, Il-10, Il-12p40, ICE, cox-2, cox-1 and mmp-3) a cell growth and differentiation panel (c-fos, c-jun and STAT3), a toxicity panel (SOD-1, TACE, GR, HSP70, GST, c-fos, c-jun, INOS) and a liver metabolism panel (INOS, cyp-a and u-pa). Other panels include skin response or prostate cancer or endothelial/cardiovascular response panels or cell growth or differentiation or liver metabolism panels. Although provided as examples, the above panels are not intended to be limiting.

For measuring the amount of a particular RNA in a sample, Source Precision used methods known to one of ordinary skill in the art to extract and quantify transcribed RNA from a sample with respect to a constituent of a panel. RNA is extracted from a sample such as a tissue, body fluid, or culture medium in which a population of a subject might be growing. For example, cells may be lysed and RNA eluted in a suitable solution in which to conduct a DNAse reaction. First strand synthesis can then be performed using a reverse transcriptase. Gene amplification, more specifically quantitative PCR assays, can then be conducted and the gene of interest size calibrated against a marker such as 18S rRNA (Hirayama et al., Blood 92, 1998: 46-52). Samples are measured in multiple duplicates for example, 4 replicates. Relative quantitation of the mRNA is determined by the difference in threshold cycles between the size marker and the gene of interest. Quantitative PCR can be, for example, performed using amplification, reporting agents and instruments such as those supplied commercially by PE Biosystems (Foster City, Calif.). Given a defined efficiency of amplification of target transcripts, the point (e.g., cycle number) that signal from amplified target template is detectable may be directly related to the amount of specific message transcript in the measured sample. Similarly, other quantifiable signals such as fluorescence, enzyme activity, disintegrations per minute, absorbance, etc., when correlated to a known concentration of target templates (e.g., a reference standard curve) or normalized to a standard with limited variability can be used to quantify the number of target templates in an unknown sample.

Although not limited to amplification methods, quantitative gene expression techniques may utilize amplification of the target transcript. Alternatively or in combination with amplification of the target transcript, amplification of the reporter signal may also be used. Amplification of the target template may be accomplished by isothermic gene amplification strategies, or by gene amplification by thermal cycling such as PCR. It is desirable to obtain a definable and reproducible correlation between the amplified target or reporter and the concentration of starting templates.

It is envisaged that techniques in the art using microfluidics for example and highly sensitive markers will enable quantitation of RNA to occur directly from a single cell or lysed cell. This may rely on amplification of a marker but may not require amplification of the transcripts themselves. The amount of transcript measured for any particular locus is a data point or member of the first profile data set for a particular panel.

A first profile data set is derived from the sample, the first profile data set including a plurality of members, each member being a quantitative measure of the amount of a RNA transcribed from a gene locus, the gene locus being a constituent in a panel of constituents. A first profile data set may be obtained from a quantitative measure of the amount of a distinct RNA or protein corresponding to a gene locus.

The analyses of samples provide a library of profile data sets relating to a particular panel or series of panels. These profile data sets may be stored as records in a library for use as baseline profile data sets. As the term “baseline” suggests, the stored baseline profile data sets serve as comparators for providing a calibrated profile data set that is informative about a homeopathic preparation. It is anticipated that many baseline profile data sets will be stored in libraries and classified in a number of cross-referential ways.

Where quality control for a newly manufactured product is being determined, the baseline data set might correspond with a gold standard for that product. However, any suitable normalization techniques may be employed.

A calibrated profile data set may be described as a function of a member of a first profile data set and a corresponding member of a baseline profile data set for a given gene locus in a panel.

The function relating the baseline and profile data sets is preferably a ratio expressed as a logarithm. The calibrated profile data set may be expressed in a spreadsheet or represented graphically for example, in a bar chart or tabular form but may also be expressed in a three dimensional representation. Preferably the particular gene is itemized on the x-axis and the logarithmic scale is on the y-axis. Members of a calibrated data set may be expressed as a positive value representing a relative enhancement of gene expression or as a negative value representing a relative reduction in gene expression with respect to the baseline.

Each member of the calibrated profile data set should be reproducible within a range with respect to similar samples taken from the subject under similar conditions. For example, the calibrated profile data sets may be reproducible within one order of magnitude with respect to similar samples taken from the subject under similar conditions. More particularly, the members may be reproducible within 50% more particularly reproducible within 20%. Each member of the calibrated profile data set has a biological significance if it has a value differing by more than an amount D, where D=F(1.1)−F(0.9) and F is a second function. It is the pattern of increasing, decreasing and no change in gene expression from the plurality of gene loci examined in the panel that is used to prepare a calibrated profile set that is informative.

The numerical data obtained from quantitative gene expression and numerical data from calibrated gene expression relative to a baseline profile data set may be stored in databases or digital storage mediums and may retrieved for purposes including managing patient health care or for conducting clinical trials or for characterizing a drug. The data may be transferred in networks via the World Wide Web, email, or internet access site for example or by hard copy so as to be collected and pooled from distant geographic sites.

A descriptive record can be stored in a single or multiple databases where the stored data includes the raw gene expression data (first profile data set) prior to transformation by use of a baseline profile data set, as well as a record of the baseline profile data set used to generate the calibrated profile data set including for example, annotations regarding whether the baseline profile data set is derived from a particular signature panel and any other annotation that facilitates interpretation and use of the data.

Because the data is in a universal format, data handling may readily be done with a computer. The data is organized so as to provide an output optionally corresponding to a graphical representation of a calibrated data set. For example, a distinct sample derived from a subject being at least one of RNA or protein may be denoted as Pᵢ. The first profile data set consists of Mⱼ where Mⱼ is a quantitative measure of a distinct RNA or protein constituent. The record Rᵢ is a ratio of M and P and may be annotated with additional data.

The above described data storage on a computer may provide the information in a form that can be accessed by a user. Accordingly, the user may load the information onto a second access site including downloading the information. However, access may be restricted to users having a password or other security device so as to protect the medical records contained within. A feature of this method is the ability of a user to add new or annotated records to the data set.

The graphical representation of calibrated profile data sets pertaining to a homeopathic preparation provides an opportunity for standardizing a product by means of the calibrated profile, more particularly a signature profile.

The method may also be implemented as a computer program product for use with a computer system. The product may include program code for deriving a first profile data set and for producing calibrated profiles. Such implementation may include a series of computer instructions fixed either on a tangible medium, such as a computer readable media (for example, a diskette, CD-ROM, ROM, or fixed disk), or transmittable to a computer system via a modem or other interface device, such as a communications adapter connected to a network over a medium. The medium may be either a tangible medium (for example, optical or analog communications lines) or a medium implemented with wireless techniques (for example, microwave, infrared or other transmission techniques). The series of computer instructions preferably embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (for example, shrink wrapped software), preloaded with a computer system (for example, on system ROM or fixed disk), or distributed from a server or electronic bulletin board over the network (for example, the Internet or World Wide Web). In addition, a computer system is further provided including derivative modules for deriving a first data set and a calibration profile data set.

As an example of obtaining a measure characteristic, human blood can be obtained by venipuncture and prepared for assay by aliquoting samples for baseline, no stimulus, and stimulus with sufficient volume for at least three time points. Typical stimuli include lipopolysaccharide (LPS), phytohemagglutinin (PHA) and heat-killed staphylococci (HKS) or carrageean and can be used individually (typically) or in combination. The aliquots of heparinized, whole blood can be mixed without stimulus and held at 37° C. in an atmosphere of 5% CO₂ for 30 minutes. Stimulus can be added at varying concentrations, mixed and held loosely capped at 37° C. for 30 min. The homeopathic preparation can be added at this point and held for varying times depending on the expected pharmacokinetics. At defined times, cells are collected by centrifugation, the plasma removed and RNA extracted by various standard means.

Nucleic acids, RNA and or DNA are purified from cells, tissues or fluids of the test population or indicator cell lines. RNA is preferentially obtained from the nucleic acid mix using a variety of standard procedures (or RNA Isolation Strategies, pp.55-104, in RNA Methodologies. A laboratory guide for isolation and characterization, 2nd edition, 1998, Robert E. Farrell, Jr., Ed., Academic Press); in the present use using a filter-based RNA isolation system from Ambion (RNAqueous™, Phenol-free Total RNA Isolation Kit, Catalog #1912, version 9908; Austin, Tex.). Specific RNAs are amplified using message specific primers or random primers. The specific primers are synthesized from data obtained from public databases (e.g., Unigene, National Center for Biotechnology Information, National Library of Medicine, Bethesda, Md.), including information from genomic and cDNA libraries obtained from humans and other animals. Primers are chosen to preferentially amplify from specific RNAs obtained from the test or indicator samples, see, for example, RT PCR, Chapter 15 in RNA Methodologies. A laboratory guide for isolation and characterization, 2^{nd} edition, 1998, Robert E. Farrell, Jr., Ed., Academic Press; or Chapter 22 pp.143-151, RNA isolation and characterization protocols, Methods in molecular biology, Volume 86, 1998, R. Rapley and D. L. Manning Eds., Human Press, or 14 in Statistical refinement of primer design parameters, Chapter 5, pp.55-72, PCR applications: protocols for functional genomics, M. A. Innis, D. H. Gelfand and J. J. Sninsky, Eds.,1999, Academic Press). Amplifications are carried out in either isothermic conditions or using a thermal cycler (for example, a ABI 9600 or 9700 or 7700 obtained from PE Biosystems, Foster City, Calif.; see Nucleic acid detection methods, pp. 1-24, in Molecular methods for virus detection, D. L. Wiedbrauk and D. H., Farkas, Eds., 1995, Academic Press). Amplified nucleic acids are detected using fluorescent-tagged detection primers (see, for example, Taqman™ PCR Reagent Kit, Protocol, part number 402823 revision A, 1996, PE Applied Biosystems, Foster City Calif.) that are identified and synthesized from publicly known databases as described for the amplification primers. Amplified DNA can be detected and quantified using the ABI Prism 7700 Sequence Detection System obtained from PE Biosystems (Foster City, Calif.). Amounts of specific RNAs contained in the test sample or obtained from the indicator cell lines can be related to the relative quantity of fluorescence observed (see for example, Advances in quantitative PCR technology: 5′ nuclease assays, Y. S. Lie and C. J. Petropolus, Current Opinion in Biotechnology, 1998, 9:43-48, or Rapid thermal cycling and PCR kinetics, pp. 211-229, chapter 14 in PCR applications: protocols for functional genomics, M. A. Innis, D. H. Gelfand and J. J. Sninsky, Eds., 1999, Academic Press.)

Other DNA, RNA and protein isolation and sequencing methods are well known to those skilled in the art. Examples of such well known techniques can be found in Molecular Cloning: A Laboratory Manual 2nd Edition, Sambrook et al, Cold Spring Harbor, N.Y. (1989); Hanspeter Saluz and J. P. Jost, A Laboratory Guide to Genomic Sequencing: The Direct Sequencing of Native Uncloned DNA (Biomethods Vol 11, Birkhauser (1988); and B. Roe et al., DNA Isolation and Sequencing, Wiley (1996). Examples of conventional molecular biology techniques include, but are not limited to, in vitro ligation, restriction endonuclease digestion, PCR, cellular transformation, hybridization, electrophoresis, DNA sequencing, cell culture, and the like. Specific kits and tools available commercially for use in the present invention include, but are not limited to, those useful for RNA isolation, PCR cDNA library construction, retroviral expression libraries, vectors, gene expression analyses, protein antibody purification, cytotoxicity assays, protein expression and purification, and high throughput plasmid purification (see, e.g., CLONTECHniques product catalog, XIII(3), 1-32 (1998) or www.clontech.com; Atlas™ cDNA Expression Assays product catalog (2000; STGMA® product catalog (2000)).

For discussions, methodologies and applications of oligonucleotide arrays, microarrays, DNA chips or biochips, see, for example, U.S. Pat. Nos. 5,445,934, 5,605,662, 5,631,1.34, 5,736,257, 5,741,644, 5,744,305, 5,795,714; Schena, et al., Parallel human genome analysis: Microarray-based expression monitoring of 1000 genes, Proc. Natl. Acad. Sci. USA 93, 10614-10619 (1996); DeRisi et al., Exploring the Metabolic and Genetic Control of Gene Expression on a Genomnic Scale, Science 278, 680-686 (1997); Wodicka, et al., Genome-wide Expression Monitoring in Saccslaromyces cerevisiae, Nature Biotechnology 15, 1359-1367 (1997); Pardee, Complete Genome Expression Monitoring: The Human Race, Nature Biotechnology 2015, 1343-1344 (1997); Schafer et al., DNA Variation and the Future of Human Genetics, Nature Biotechnology 16, 33-39 (1998); DeRisi et al., Use of a cDNA Microarray to Analyze Gene Expression Patterns in Human Cancer, Nature Genetics 14, 457-460 (1996); Heller et al., Discovery and Analysis of Inflammatory Disease Related Genes Using cDNA Microarrays, Proc. Natl. Acad. Sci. USA 94, 2150-2155 (1997); Marshall et al., DNA Chips: An Array of Possibilities, Nature Biotechnology 16, 27-31 (1998); Schena et al., Microarrays: Biotechnology's Discovery Platform for Functional Genomics, Tibtech 16, 301-306 (1998); Ramsay, DNA Chips: State-of-the art, Nature Biotechnology 16, 40-44 (1998); Chee et al., Accessing Genetic Information with High-Density DNA Arrays, Science 274, 610-614 (1996); and Chen et al., Profiling Expression Patterns and Isolating Differentially Expressed Genes by cDNA Microarray System with Colorimetry Detection, Genomics 50, 1-12 (1998); P. Andrew Outinen et al., Characterization of the stress-inducing effects of homocysteine, Biochem. J. 332, 213-221 (1998); and Gelbert et al., Will genetics really revolutionize the drug discovery process, Curr Opin Biotechnol 8(6), 669-674 (1997).

Other, more specific, references applicable to the instant invention include, but are not limited to, those addressing the expression technologies, such as ESTs (see, e.g., Michael R. Fannon, Gene expression in normal and disease states-identification of therapeutic targets, TIBTECH 14, 294-298 (1996)); the generation of protein profiles (see, e.g., Robinson et al., A Tyrosine Kinase Profile of Prostate Carcinoma, Proc Natl. Acad. Sci. USA 93, 5958-5962 (1996)); chemical and spectroscopic methods for identifying components of herbal compositions (Kojima et al., Saponins from Gliricidia sepium, Phytochemistry 48(5), 885-888 (1998)); the determination of functional antigens (see, e.g., Aris Persidis, Functional antigenics, Nature Biotechnology 16, 305-:307 (1998)); HPLCs (see, e.g., Milton T. W. Hearn (Editor), HPLC of Proteins. Pepties and Polynucleotides: Contemporary Topics and Applications (Analytical Techniques in Clinical Chemistry and Laboratory Manual), VCH Pub. (1991); electrophoresis (see, e.g., Westermeier et al., Electrophoresis in Practice: A Guide to Methods and Applications of DNA and Protein Separations. John Wiley & Sons (1997)); and cross-reactivity marker assays (see, e.g., Irving Millman et al., Woodchuck Hepatitis Virus: Experimental Infection and Natural Occurrence, Hepatology 4(5):817-823 (1984)). The use of structural genomics for solving the structures of all the proteins encoded for in completed genomes, wherein the methodology includes high-throughput direct structure determinations and computational methods, is discussed by Terry Gaasterland, Structural genomics: Bioinformatics in the driver's seat, Nature Biotechnology 16, 625-627. For bioinformatics methodologies, see, for example, Andreas Baxevanis (Editor), Bioinformatics A Practical Guide to the Analysis of Genes and Proteins, John Wiley & Sons (1998) and Luke Alphey, DNA Sequencing: Sequencing: From Experimental Methods to Bioinformatics (Introduction to Biotechniques Series), Springer Verlag (1997).

Cytogenetic parameters include, but are not limited to, karyotype analyses (e.g., relative chromosome lengths, centromere positions, presence or absence of secondary constrictions), ideograms (i.e., a diagrammatic representation of the karyotype of an organism), the behavior of chromosomes during mitosis and meiosis, chromosome staining and banding patterns, DNA-protein interactions (also known as nuclease protection assays), neutron scattering studies, rolling circles (A. M. Diegelman and E. T. Kool, Nucleic Acids Res 26(13):3235-3241 (1998); Backert et al., Mol. Cell. Biol. 16(11):6285-6294 (1996); Skaliter et al., J. Viol. 70(2):1132-1136 (1996); A. Fire and S. Q. Xu, Proc. Natl. Acad. Sci. USA 92(10):4641-4645 (1995)), and autoradiography of whole nuclei following incubation with radiolabelled ribonucleotides.

Biochemical parameters include, but are not limited to, specific pathway analyses, such as signal transduction, protein synthesis and transport, RNA transcription, cholesterol synthesis and degradation, glucogenesis and glycolysis.

The level of gene expression then constitutes the measure characteristic of the composition. This measure characteristic based on gene expression can then be compared with the measure characteristic of other compositions. The comparison of the measure characteristic will then identify whether the two compositions compared are equivalent or substantially equivalent. Equivalent compositions are compositions which have equivalent or substantially equivalent measure characteristics of gene expression. Thus, by having am effective method for determining the equivalence of two chemical compounds, such as a homeopathic preparation, quality control can be maintained.

A method is also provided for ensuring quality control between a homeopathic preparation and a baseline profile for that preparation. A baseline profile is provided by obtaining a measure characteristic for one or more homeopathic preparations, preferably more than one. Most; preferably, the baseline profile has been formed by the accumulation of many characteristic profiles for homeopathic preparation demonstrated to be pure and/or therapeutically effective. A measure characteristic is then obtained for the homeopathic preparation to be tested for quality control. The measure characteristic for the homeopathic preparation to be tested for quality control is then compared to the baseline profile. A large difference between the measure characteristic for the homeopathic preparation and the baseline profile indicates a difference between the homeopathic preparation and the baseline profile.

In a preferred embodiment, the at least one homeopathic preparation and the homeopathic preparation to be tested are derived from a single source type. By single source type, it is meant that the homeopathic preparations to be compared are derived from the same substance in order to make the mother tincture. This substance used to make the mother tincture may or may not be obtained from the same source.

For example, two homeopathic preparations can be compared derived from calcium carbonate, a mineral frequently used in preparing homeopathic preparations. Calcium carbonate is often obtained from oyster shells. Thus, the homeopathic preparations to be compared can be obtained from the same or different oyster shells.

If the homeopathic preparations to be compared are derived from substances obtained from different sources, each of the homeopathic preparations will necessarily be derived from a different mother tincture. However, it may be desirable to compare two different homeopathic preparations derived from the same mother tincture.

As discussed above, homeopathic preparations are prepared with different potencies. According to homeopathy, the higher the dilution, the higher the potency. Therefore, it is preferred that the homeopathic preparations to be compared are of equal potency.

The following examples are provided to assist in a further understanding of the invention. The particular materials and conditions employed are intended to be further illustrative of the invention and are not limiting upon the reasonable scope thereof.

### EXAMPLE 1

This example demonstrates gene expression as a tool to obtain measure characteristic for homeopathic products.

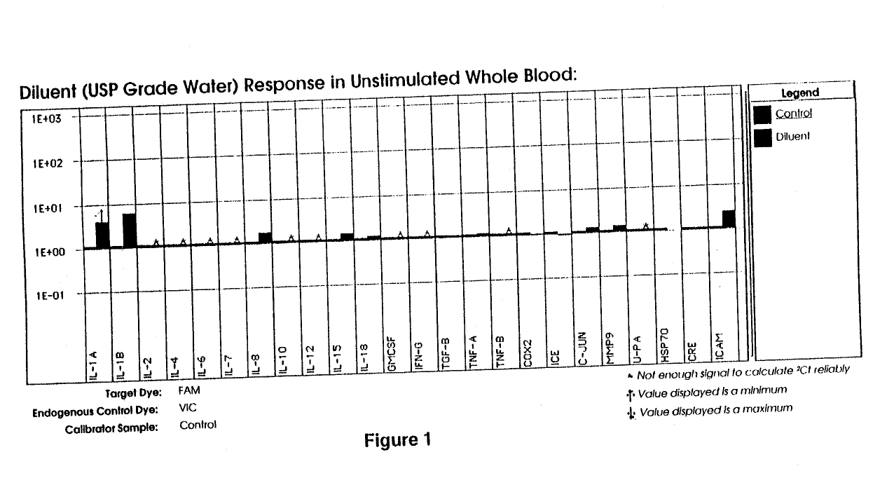

To establish baseline parameters, the gene expression response of select Arnica homeopathic preparations were examined in unstimulated human blood cells using Source's Precision Profile™ for Inflammation. Since homeopathic concepts deal with high chemical dilution and attenuation effects, the use of appropriate controls is important. USP water is the compendial (HPUS) standard diluent and used by used by Boericke & Tafel (Santa Rosa, Calif.) and therefore, serves as the appropriate control. The gene expression effects of the diluent (USP water) in unstimulated whole blood are insignificant as shown in FIG. 1.

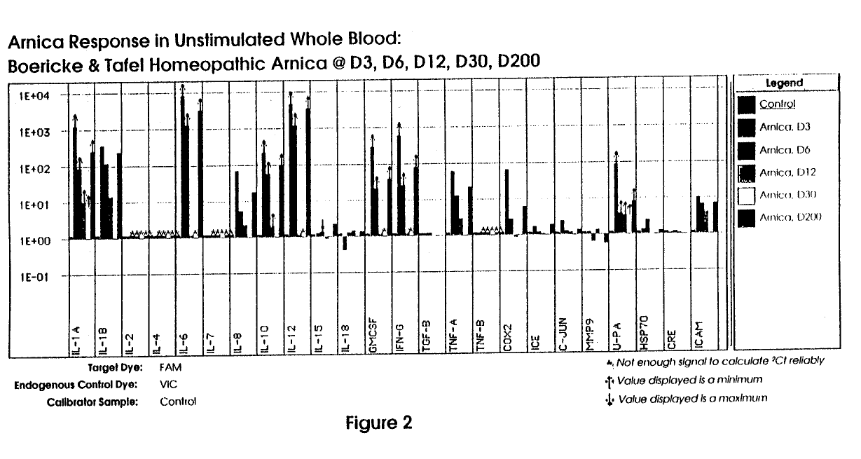

The gene expression response for Boericke & Tafel (Santa Rosa, Calif.) homeopathic Arnica 3X, 6X, 12X, 30X and 200X is shown in FIG. 2. Presence of an asterisk (*) in figures indicates signal unchanged from calibrator, therefore, no differential gene expression is observed. Presence of an upward arrow indicates the gene expression value is a minimum. Presence of a downward arrow indicates the gene expression value is a maximum.

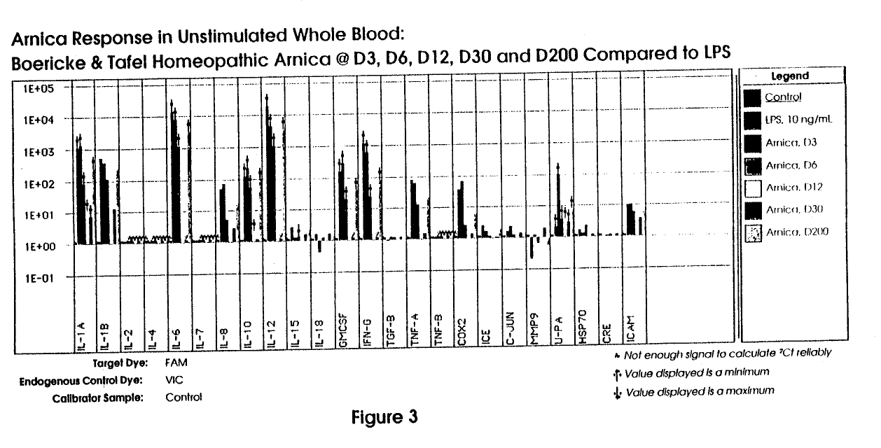

FIG. 2 demonstrates a clear up regulation of both pro-inflammatory (see IL-1α, IL-1B, IL-8, IL-12, TNF-α and cox-2) and anti-inflammatory (see IL-10) mediators. Gene expression response decreases going from 3X, 6X, 12X, and 30X. The gene expression patterns exhibited by these preparations resembles the gene expression response pattern of endotoxin (LPS) in unstimulated whole blood. This comparison is demonstrated in FIG. 3. By this observation, it was determined that the decrease in gene expression was consistent with the decrease in endotoxin levels. The presence of an endotoxin in the these preparations explains the decreasing gene expression. On the other hand, FIG. 2 demonstrates a significant increase in the magnitude of gene expression for attenuation to 200X, which it was found was not contaminated with endotoxin.

These results confirm the ability of the gene panel to establish a measure characteristic for not only the specific variables designed into the test, but also unknown or unexpected results, possibly due to a particular contaminant.

### EXAMPLE 2

Having established baseline parameters of gene expression response for homeopathic Arnica preparations as set forth in Example 1, the method of the invention was used to compare preparations by different manufacturers, as well as different lots of the same preparation by the same manufacturer.

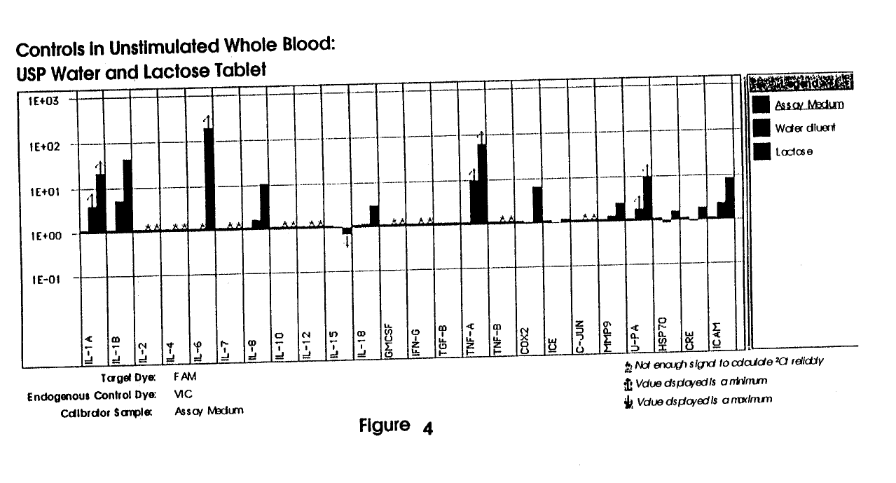

USP water and a lactose tablet serve as controls. As FIG. 4 shows, the USP water diluent exhibits a slight up regulation of key pro-inflammatory mediators (see IL-1α, IL-1B, TNF-α). Moreover, the lactose tablet exhibits considerable gene expression response in key pro-inflammatory mediators (see IL-1α, IL-1B, IL-6, IL-8, -TNF-α and cox-2). This demonstrated up regulation is considered when comparing the gene expression profiles for the homeopathic preparations.

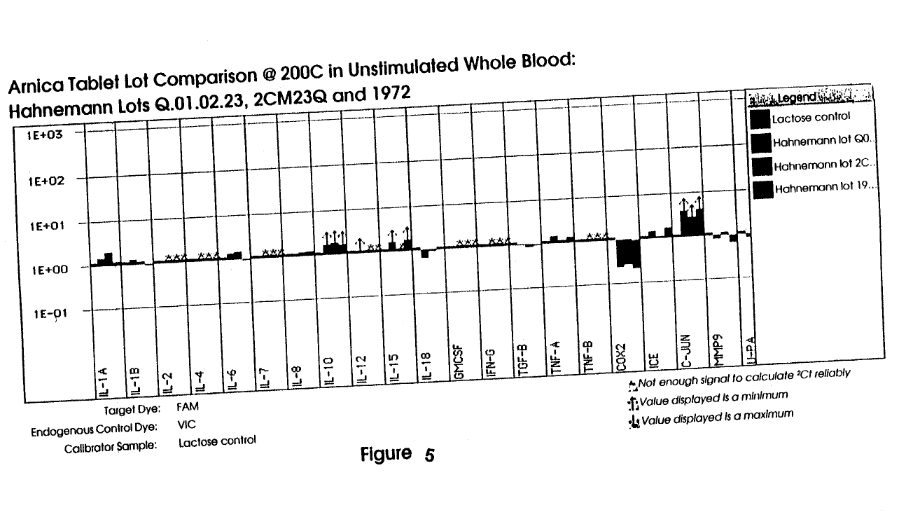

FIG. 5 demonstrates the comparison of three lots of a preparation from the same manufacturer. Three 200C Arnica lots from Hahnemann Laboratories, Inc. (San Rafael, Calif.) show weak, but near identical gene expression responses in unstimulated whole blood as shown in FIG. 5. Such gene expression responses can be utilized and compared, for example, in quality control.

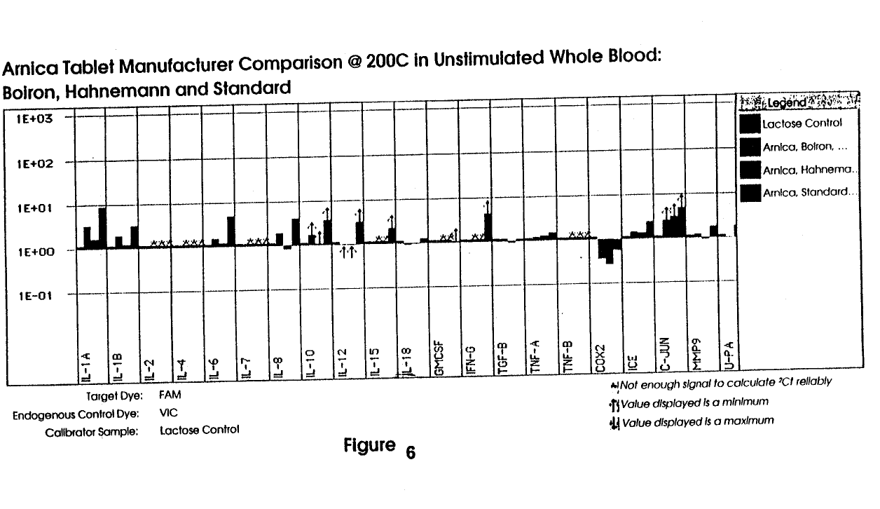

FIG. 6 demonstrates a comparison between three different manufacturers of homeopathic preparations. FIG. 6 demonstrates a comparison of gene expression profiles generated for a 200C Arnica preparation from each of Boiron Laboratories (France), Hahnemann Laboratories, Inc. (San Rafael, Calif.) and Standard Homeopathic (Los Angeles, Calif.). Certain genes within the profile demonstrate essentially the same expression for each of the preparations, e.g. IL-1B, IL-4, IL-7, GMCSF, TNF-B. Other genes demonstrate a difference in gene expression between the preparations from the various manufacturers, e.g. IL-1A, IL-1B, IL-6, IL-8, COX2, ICE, C-JUN. The ability to detect such differences among preparations can be utilized, for example, as an important research or quality control tool.

### EXAMPLE 3

This example demonstrates the method of the invention on homeopathic preparations, the manufacture of which requires succussion, versus unsuccussed preparations. This example also further demonstrates the ability to utilize the invention for highly dilute (attenuated) preparations.

Succussed preparations were obtained commercially (Boericke & Tafel). Unsuccussed preparations were made by serial dilutions of 1X (mother tincture) Arnica (Boericke & Tafel) stock at Source Precision Medicine (Boulder, Colo.).

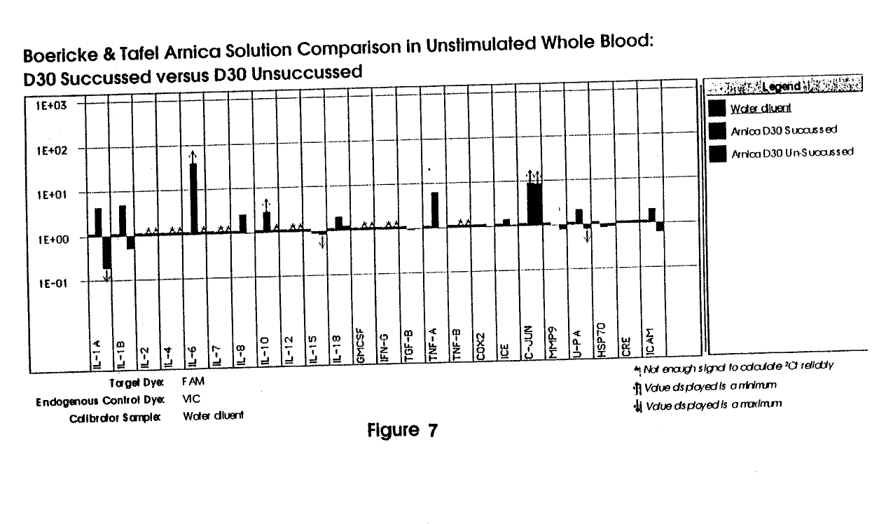

FIG. 7 demonstrates a significant increase in gene expression for the 30X succussed Arnica preparation versus the 30X unsuccussed Arnica preparation in unstimulated whole blood. (See IL-1α, IL-1B, IL-6, IL-8, IL-10, TNF-α, u-pa and ICAM).

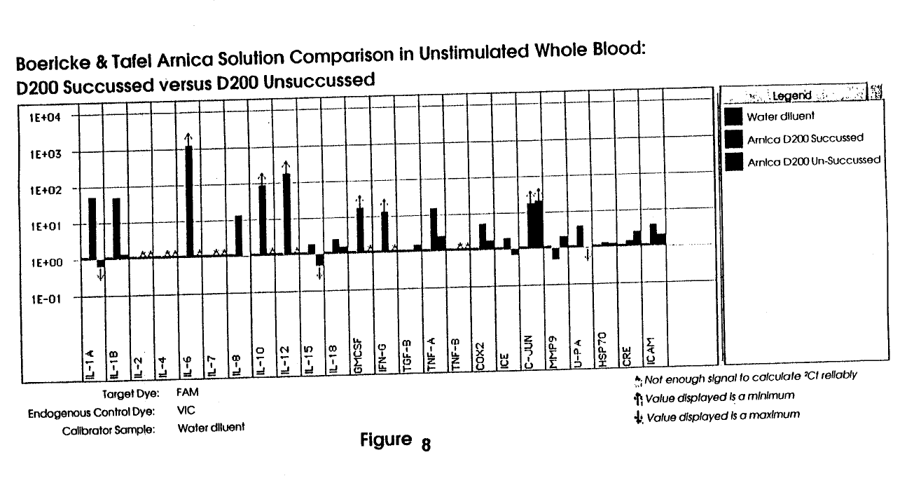

FIG. 8 demonstrates that the succussed 200X Arnica preparation in unstimulated whole blood shows a much greater level of gene expression as compared to the 200X unsuccussed Arnica non-homeopathic preparation. (See IL-1A, IL-1B, IL-6, IL-8, IL-10, IL-12, GMCSF, IFN-G, TNF-A).

Therefore, the results in general demonstrate that gene expression can be utilized to obtain a measure characteristic for homeopathic preparations, even for highly dilute (attenuated) homeopathic preparations.

What is claimed is:

1. A method for obtaining a measure characteristic of a homeopathic preparation, the method comprising:
a) contacting the homeopathic preparation with at least one gene in a gene expression system; and
b) measuring expression of said at least one gene;
wherein the level of expression constitutes the measure characteristic.

2. A method as described in claim 1 wherein said gene expression system is a gene expression microarray.

3. A method as described in claim 1 wherein said gene is selected from the group consisting of IL-1alpha; IL-1beta; IL-2; IL-4; IL-6; IL-7; IL-8; IL-10; IL-12p40; IL-15; IL-18; GM-CSF; IFN-gamma; TGF-beta; TNF-alpha; ICE; c-juh; MMP-9; u-pa; HSP-70; cre; ICAM; Cox-2; Cox-1, or a combination thereof.

4. A method for ensuring quality control of a homeopathic preparation, the method comprising:
obtaining at least one measure characteristic for one or more homeopathic preparations according to claim 1 to obtain a baseline profile;
obtaining a measure characteristic for a second homeopathic preparation according to claim 1; and
determining the difference in measure characteristic between the baseline profile and second homeopathic preparation.

5. A method as described in claim 4 wherein said one or more homeopathic preparations and said second homeopathic preparation are derived from a single source type.

6. A method as described in claim 4 wherein said one or more homeopathic preparations and said second homeopathic preparation have each have an equivalent potency.

7. A method as described in claim 4 wherein said one or more homeopathic preparations and said second homeopathic preparation are derived from different mother tinctures.

8. A method as described in claim 4 wherein said one or more homeopathic preparations and said second homeopathic preparation are derived from a single mother tincture.
